(19) United States
(12) Patent Application Publication
(10) Pub. No.: US 2025/0073019 A1
(43) Pub. Date: Mar. 6, 2025

(54) MEDICAMENT RESERVOIR FOR AN INTRAOCULAR LENS, AND INTRAOCULAR LENS HAVING SUCH A MEDICAMENT RESERVOIR

(71) Applicant: Carl Zeiss Meditec AG, Jena (DE)

(72) Inventors: Niklas Damm, Oberkochen (DE); André Wolfstein, Oberkochen (DE)

(21) Appl. No.: 18/948,168

(22) Filed: Nov. 14, 2024

Related U.S. Application Data
(63) Continuation of application No. PCT/EP2023/062818, filed on May 12, 2023.

(30) Foreign Application Priority Data
May 20, 2022 (DE) 10 2022 112 804.0

(51) Int. Cl. A61F 2/16

(52) U.S. Cl. CPC A61F 2/1601; A61F 2250/0068

(57) ABSTRACT

A medicament reservoir for an intraocular lens has a medicament, a through-hole, a first medicament reservoir clearance which communicates with the through-hole, a first web which delimits the first medicament reservoir clearance, a first end face which delimits the through-hole, and a second end face which is disposed so as to face away from the first end face and to delimit the through-hole. The through-hole in a region of the first end face has a first longitudinal end and in a region of the second end face has a second longitudinal end. The medicament reservoir has a displacement direction oriented from the first longitudinal end to the second longitudinal end. The first medicament reservoir clearance is formed such that the through-hole is accessible from outside the medicament reservoir via the first medicament reservoir clearance in a medicament reservoir radial direction in terms of the displacement direction.

### CROSS-REFERENCE TO RELATED APPLICATIONS

This application is a continuation application of international patent application PCT/EP2023/062818, filed May 12, 2023, designating the United States and claiming priority from German application 10 2022 112 804.0, filed May 20, 2022, and the entire content of both applications is incorporated herein by reference.

### TECHNICAL FIELD

The disclosure relates to a medicament reservoir for an intraocular lens and to an intraocular lens having such a medicament reservoir.

### BACKGROUND

In cataract treatment of an eye, a natural lens is replaced by an artificial intraocular lens. For this purpose, only a small incision is usually made in the cornea of the eye, the incision being large enough to allow a tip of an injector to be introduced into the eye through the incision. After the incision has been made in the cornea, the natural lens of the eye is usually comminuted by phacoemulsification and then suctioned out of the capsular bag of the eye. Thereafter, the intraocular lens is inserted into the eye by means of the injector.

After cataract treatment, drugs such as, for example, antibiotics and/or anti-inflammatory drugs are usually delivered into the eye. For example, a medicament reservoir which is introduced into the eye together with the intraocular lens can be attached to the intraocular lens. If the intraocular lens is inserted into the eye via the tip, the medicament reservoir should not be released from the intraocular lens. US 2021/0267751 A1 discloses an intraocular drug delivery platform. US 2014/0148900 A1 discloses intraocular devices to which a drug delivery construct is attached.

### SUMMARY

It is an object of the disclosure to provide a medicament reservoir and an intraocular lens having the medicament reservoir, wherein the medicament reservoir, when the medicament reservoir is attached to the intraocular lens, is not released from the intraocular lens.

A medicament reservoir according to the disclosure for an intraocular lens has a medicament, a through-hole, a first medicament reservoir clearance which communicates with the through-hole, a first web which delimits the first medicament reservoir clearance, a first end face which delimits the through-hole, and a second end face which is disposed so as to face away from the first end face and to delimit the through-hole, wherein the through-hole in a region of the first end face has a first longitudinal end and in a region of the second end face has a second longitudinal end, wherein the medicament reservoir has a displacement direction which is oriented from the first longitudinal end to the second longitudinal end, wherein the first medicament reservoir clearance is formed such that the through-hole is accessible from outside the medicament reservoir by way of the first medicament reservoir clearance in a medicament reservoir radial direction in terms of the displacement direction. The medicament reservoir is able be coupled to a haptic arm of an intraocular lens in that a longitudinal end of the haptic arm, which is disposed so as to face away from an optical body of the intraocular lens, is disposed in the through-hole, and the medicament reservoir is subsequently displaced in the displacement direction and closer toward the optical body. The medicament reservoir can be attached particularly firmly to the haptic arm in that part of the haptic arm is subsequently introduced into the first medicament reservoir clearance, for example in that the haptic arm has a protrusion which is specified to engage in the first medicament reservoir clearance. Thus, there is a low probability that the medicament reservoir will be released from the intraocular lens, especially if the intraocular lens is inserted into the capsular bag of an eye by way of a tip of an injector.

The displacement direction is preferably disposed parallel to a normal of the first end face and/or parallel to a normal of the second end face.

It is preferred that the first web forms part of the first end face. Particularly preferably, the first web delimits the first medicament reservoir clearance in a direction which is oriented counter to the displacement direction.

It is preferred that the second end face is not formed in a region of the first medicament reservoir clearance, so that the first medicament reservoir clearance is accessible from outside the medicament reservoir in the displacement direction. When the haptic arm is disposed in the through-hole, this makes it possible to introduce the haptic arm into the first medicament reservoir clearance by a pivoting movement of the medicament reservoir.

The medicament reservoir preferably has a second medicament reservoir clearance which communicates with the through-hole, and a second web which delimits the second medicament reservoir clearance, wherein the second medicament reservoir clearance is formed such that the through-hole is accessible from outside the medicament reservoir by way of the second medicament reservoir clearance in a further medicament reservoir radial direction in terms of the displacement direction. Owing to the fact that the haptic arm is disposed in the second medicament reservoir clearance, in particular in that a further protrusion of the haptic arm engages in the second medicament reservoir clearance, the medicament reservoir can be attached even more firmly to the haptic arm.

The first medicament reservoir clearance and the second medicament reservoir clearance are preferably spaced apart from one another.

The second web preferably forms part of the second end face. Particularly preferably, the second web delimits the second medicament reservoir clearance in the displacement direction.

It is preferred that the first end face is not formed in a region of the second medicament reservoir clearance, so that the second medicament reservoir clearance is accessible from outside the medicament reservoir counter to the displacement direction. This makes it advantageously possible to introduce the haptic arm simultaneously into the first medicament reservoir clearance and the second medicament reservoir clearance by way of the pivoting movement.

It is preferred that the medicament reservoir is specified to continuously dispense the medicament. In particular, the medicament reservoir can be specified to be biodegradable. For this purpose, the medicament reservoir can have a matrix into which the medicament can be introduced, including a copolymer formed by a first monomer and a second monomer. The first monomer can be a caprolactone, and the second monomer can be selected from the group including lactide, glycolide and/or trimethylene carbonate. An example of a matrix in which the medicament can be introduced and which is not biodegradable is a polymerized hydroxyethyl methacrylate.

The medicament can include, for example, an antibiotic such as moxifloxacin, and/or a steroidal anti-inflammatory drug such as, for example, dexamethasone, and/or a non-steroidal anti-inflammatory drug such as, for example, a non-steroidal anti-rheumatic drug such as, for example, diclofenac. For example, the medicament can additionally or alternatively include a diagnostic substance, such as a contrast agent.

The medicament reservoir can, for example, include a single material which includes the matrix with the medicament introduced in the matrix, or consists of the matrix with the medicament introduced in the matrix.

The medicament reservoir preferably includes a first material which includes the matrix with the medicament introduced in the matrix, and a second material which is different from the first material and forms the first web and in particular the second web. The second material is preferably not biodegradable. For this purpose, the second material can be selected from the group including polymethyl methacrylate, polymerized hydroxyethyl methacrylate, polypropylene, silicone, acrylate copolymer.

The medicament reservoir preferably has a first cover plate which is formed by the second material and forms the first end face. Moreover, the medicament reservoir can have a second cover plate which is formed by the second material and forms the second end face.

The intraocular lens according to the disclosure has an optical body, a haptic arm which is fastened to the optical body, and the medicament reservoir according to the disclosure or a preferred embodiment of the medicament reservoir according to the disclosure, wherein the haptic arm is disposed in the through-hole and is disposed in the first medicament reservoir clearance.

It is preferred that the haptic arm is of a curved configuration. The haptic arm is particularly preferably configured to be C-shaped or J-shaped.

The medicament reservoir is preferably fastened to the haptic arm by means of a form fit and/or an interference fit. As a result, the medicament reservoir is particularly firmly disposed on the haptic arm.

### BRIEF DESCRIPTION OF DRAWINGS

The invention will now be described with reference to the drawings wherein:

FIG. 1 shows a top view of an intraocular lens according to the disclosure, which is drawn so as to be disposed in a capsular bag;

FIG. 2 shows a perspective view of a medicament reservoir not associated with the disclosure;

FIG. 3 shows a perspective view of a first medicament reservoir according to the disclosure;

FIG. 4 shows a perspective view of a second medicament reservoir according to the disclosure;

FIG. 5 shows a lateral view of an intraocular lens according to the disclosure;

FIG. 6 shows a haptic arm and a medicament reservoir according to the disclosure, the latter being disposed so as to be spaced apart from the haptic arm;

FIG. 7 shows the haptic arm and the medicament reservoir from FIG. 6, wherein the medicament reservoir is displaced onto the haptic arm; and,

FIG. 8 shows the haptic arm and the medicament reservoir from FIGS. 6 and 7, which are drawn so as to be disposed in a capsular bag, wherein a first web of the haptic arm is disposed in a first axial clearance of the haptic arm, and a second web of the haptic arm is disposed in a second axial clearance of the haptic arm.

### DETAILED DESCRIPTION

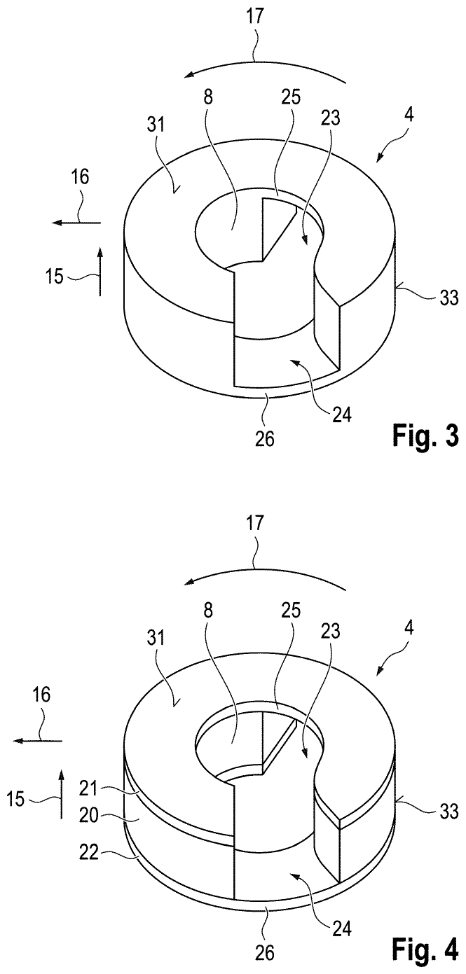

As can be seen from FIGS. 3 and 4, a medicament reservoir 4 according to the disclosure for an intraocular lens 1 has a medicament, a through-hole 8, a first medicament reservoir clearance 23 which communicates with the through-hole 8, a first web 25 which delimits the first medicament reservoir clearance 23, a first end face 31 which delimits the through-hole 8, and a second end face 32 which is disposed so as to face away from the first end face 31 and to delimit the through-hole 8. The through-hole 8 has a first longitudinal end 34 in a region of the first end face 31 and a second longitudinal end 35 in a region of the second end face 32. The medicament reservoir 4 has a displacement direction 15 which is oriented from the first longitudinal end 34 to the second longitudinal end 35. The first medicament reservoir clearance 23 is formed such that the through-hole 8 is accessible from outside the medicament reservoir 4 by way of the first medicament reservoir clearance 23 in a medicament reservoir radial direction 16 in terms of the displacement direction 15. The medicament reservoir 4 according to the disclosure differs from the medicament reservoir 4 from FIG. 2 in that the first medicament reservoir clearance 23 is not provided in the medicament reservoir 4 from FIG. 2.

The medicament reservoir 4 can have on its outside a circumferential face 33 which is disposed between the first end face 31 and the second end face 32, and in particular can be directly contiguous to the first end face 31 and the second end face 32.

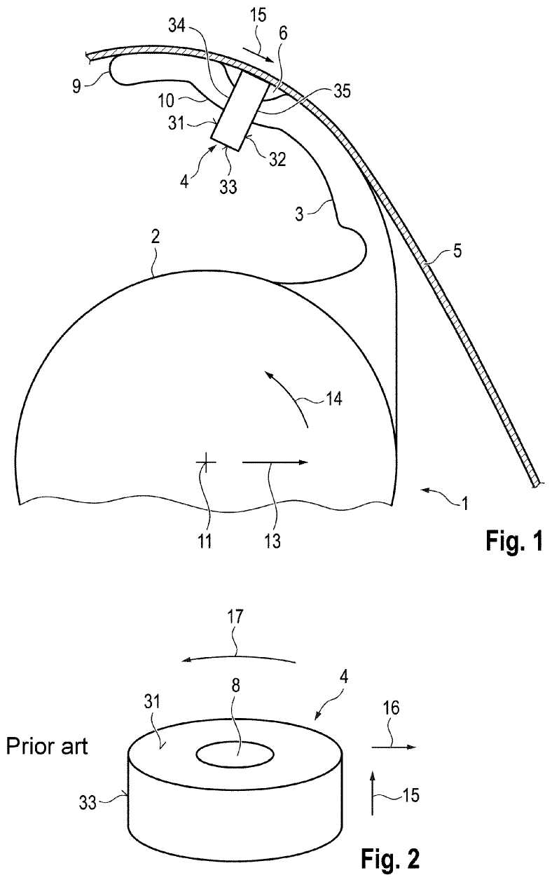

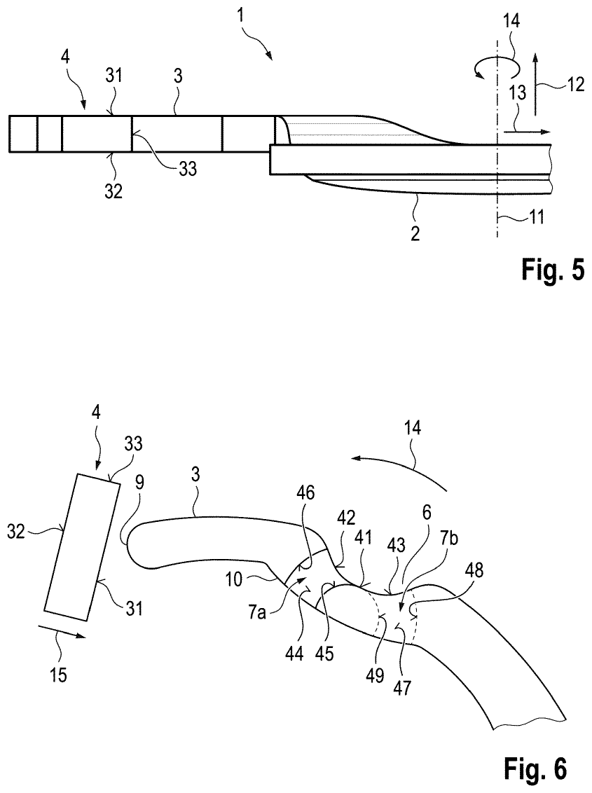

As can be seen from FIGS. 1, 5 and 8, an intraocular lens 1 according to the disclosure has an optical body 2 (cf. FIGS. 1 and 5), a haptic arm 3 which is fastened to the optical body 2, and the medicament reservoir 4, wherein the haptic arm 3 is disposed in the through-hole 8 and is disposed in the first medicament reservoir clearance 23.

The haptic arm 3 can be of a curved configuration, and in particular be configured to be C-shaped (see FIG. 1) or J-shaped.

The medicament reservoir 4 can be fastened to the haptic arm 3 by means of a form fit and/or an interference fit, for example.

FIGS. 1 and 5 show that the optical body 2 can include an optical axis 11. The haptic arm 3 can have a radial clearance 6 (cf. FIGS. 1 and 6 to 8) in which the medicament reservoir 4 is disposed and which is incorporated in a side of the haptic arm 3 that is disposed on the outside in a radial direction 13 in terms of the optical axis 11. Owing to the fact that the medicament reservoir 4 is disposed in the radial clearance 6, the medicament reservoir 4 is disposed particularly firmly on the haptic arm 3. There is therefore little possibility of the medicament reservoir 4 detaching from the haptic arm 3, especially if the intraocular lens 1 is injected via a tip of an injector into a capsular bag 5 (cf. FIGS. 1 and 8) of an eye. Owing to the fact that the radial clearance 6 is incorporated in the outer side of the haptic arm 3 in the radial direction 13, the medicament reservoir 4 does not protrude, or protrudes only slightly, outward from the haptic arm 3 in the radial orientation 13. Because the outer side of the haptic arm 3 in the radial direction 13 contacts the capsular bag 5, a decentration of an optical body 2 of the intraocular lens 1 in the eye by the medicament reservoir 4 can be reduced or even avoided.

FIGS. 1 and 8 show how the medicament reservoir 4 can be recessed in the radial clearance 6. This has the effect that, when the intraocular lens 1 is introduced into the capsular bag 5 of an eye, the medicament reservoir 4 does not cause the capsular bag 5 to bulge outward, that is, away from the optical body 2, cf. FIGS. 1 and 8.

FIG. 6 shows that the radial clearance 6 can be delimited by a first flank 41, which delimits a displacement of the medicament reservoir 4 counter to the radial direction 13, that is, toward the optical axis 11. Moreover, the radial clearance 6 can be delimited by a second flank 42 which delimits a displacement of the medicament reservoir 4 in a circumferential direction 14 in terms of the optical axis 11. Moreover, the radial clearance 6 can be delimited by a third flank 43 which delimits a displacement of the medicament reservoir 4 in a direction that is oriented counter to the circumferential direction 14.

FIGS. 3 and 4 show that the first web 25 can form part of the first end face 31. FIGS. 6 to 8 show that the haptic arm 3 can have a first axial clearance 7a in which the first web 25 is disposed and which is incorporated in a first side of the haptic arm 3 that is disposed on the outside in an axial direction 12 in terms of the optical axis 11. The second end face 32 may not be formed in a region of the first medicament reservoir clearance 23, cf. FIGS. 3 and 4, so that the first medicament reservoir clearance 23 is accessible from outside the medicament reservoir 4 in the displacement direction 15. The first web 25 can delimit the first medicament reservoir clearance 23 in a direction which is oriented counter to the displacement direction 15.

FIG. 6 shows that the first axial clearance 7a can be delimited by a first flank 44 which delimits a displacement of the medicament reservoir 4 counter to the axial direction 12. Moreover, the first axial clearance 7a can be delimited by a second flank 45 which delimits a displacement of the medicament reservoir 4 counter to the circumferential direction 14. Moreover, the first axial clearance 7a can be delimited by a third flank 46 which delimits a displacement of the medicament reservoir 4 in the circumferential direction 14.

The first flank 41 of the radial clearance 6 and the first flank 44 of the first axial clearance 7a can, for example, enclose an angle of 60° to 120°, in particular of 80° to 100° or substantially 90°.

FIGS. 3 and 4 also show that the medicament reservoir 4 can have a second medicament reservoir clearance 24 which communicates with the through-hole 8, and a second web 26 which delimits the second medicament reservoir clearance 24, wherein the second medicament reservoir clearance 24 is formed such that the through-hole 8 is accessible from outside the medicament reservoir 4 by way of the second medicament reservoir clearance 24 in a further medicament reservoir radial direction 16 in terms of the displacement direction 15. The first medicament reservoir clearance 23 and the second medicament reservoir clearance 24 can be spaced apart from one another. In particular, the first medicament reservoir clearance 23 and the second medicament reservoir clearance 24 can be disposed so that they follow the curvature of the haptic arm 3 and thus do not cause any deformation of the haptic arm 3 when the medicament reservoir 4 is attached to the haptic arm 3.

The second web 26 can form part of the second end face 32. It can moreover be seen from FIGS. 6 to 8 that the haptic arm 3 can have a second axial clearance 7b in which the second web 26 is disposed and which is offset from the first axial clearance 7a in a circumferential direction 14 in terms of the optical axis 11 and is incorporated in a second side of the haptic arm 3, which side is disposed on the outside, counter to the axial direction 12, and is disposed so as to face away from the first side. The first end face 31 may not be formed in a region of the second medicament reservoir clearance 24, so that the second medicament reservoir clearance 24 is accessible from outside the medicament reservoir 4 counter to the displacement direction 15. The second web 26 can delimit the second medicament reservoir clearance 24 in the displacement direction 15.

FIG. 6 shows that the second axial clearance 7b can be delimited by a first flank 47 which delimits a displacement of the medicament reservoir 4 in the axial direction 12. Moreover, the second axial clearance 7b can be delimited by a second flank 48 which delimits a displacement of the medicament reservoir 4 counter to the circumferential direction 14. Moreover, the second axial clearance 7b can be delimited by a third flank 49 which delimits a displacement of the medicament reservoir 4 in the circumferential direction 14.

The first flank 41 of the radial clearance 6 and the first flank 47 of the second axial clearance 7b can, for example, enclose an angle of 60° to 120°, in particular of 80° to 100° or substantially 90°.

The first axial clearance 7a can be directly contiguous to the radial clearance 6, and/or the second axial clearance 7b can be directly contiguous to the radial clearance 6 (cf. FIGS. 6 to 8). The second axial clearance 7b can be disposed so as to be spaced apart from the first axial clearance 7a in the circumferential direction 14. FIGS. 1 and 6 to 8 show that the intraocular lens 1 can have a radial protrusion 10 which in the radial direction 13 protrudes inward from the remainder of the haptic arm 3 and which, in a circumferential direction 14 with respect to the optical axis 11, is disposed in the same region as the radial clearance 6.

The first web 25 can be recessed in the first axial clearance 7a, so that the medicament reservoir 4, when viewed in the radial direction 13, can be disposed in a region of the first side so as to be flush with the haptic arm 3 (cf. FIG. 5). The second web 26 can be recessed in the second axial clearance 7b, so that the medicament reservoir 4, when viewed in the radial direction 13, can be disposed in a region of the second side so as to be flush with the haptic arm 3 (cf. FIG. 5). Owing to this fact, it can be particularly reliably avoided that the medicament reservoir 4 is released from the haptic arm 3 when injecting the intraocular lens 1. When folding the intraocular lens 1 prior to injecting the intraocular lens 1, it is also easily possible to convey the haptic arm 3 onto the optical body 2 and fold the optical body 2 around the haptic arm 3.

The displacement direction 15 can be oriented in the direction in which the medicament reservoir 4 is to be displaced in order to displace the medicament reservoir 4 into the radial clearance 6. It is conceivable that the displacement direction 15 is disposed parallel to a normal of the first end face 31 and/or parallel to a normal of the second end face 32. The first end face 31 can form the longitudinal end of the medicament reservoir 4 located in the displacement direction 15, and the second end face 21 can form the longitudinal end of the medicament reservoir 4 located in a direction which is oriented counter to the displacement direction 15.

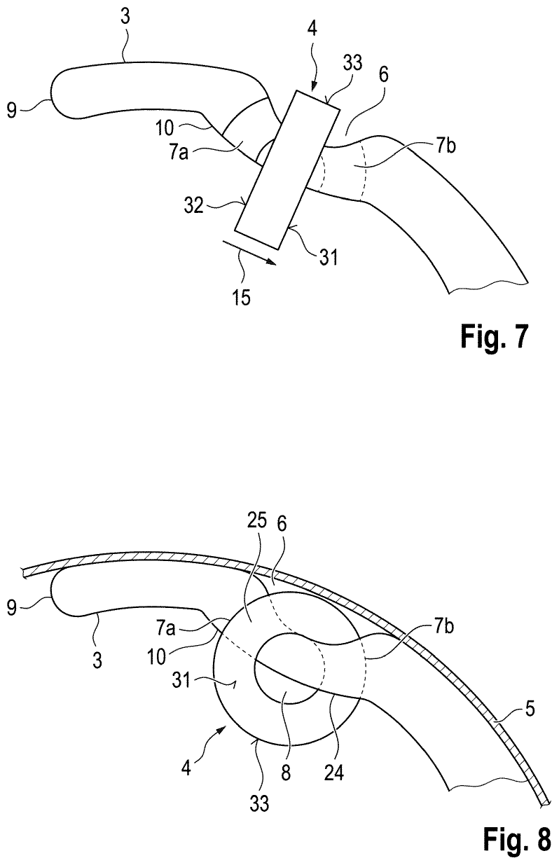

FIGS. 6 to 8 show how the medicament reservoir 4 can be attached to the haptic arm 3. First, the medicament reservoir 4 is disposed so as to be spaced apart from the haptic arm 3. Subsequently, a longitudinal end 9 of the haptic arm 3, that is disposed so as to face away from the optical body 2, can first be disposed in the through-hole 8. By displacing the medicament reservoir 4 in the displacement direction 15, the medicament reservoir 4 can be displaced until the medicament reservoir 4 reaches the radial clearance 6 (cf. FIG. 7). In the first embodiment of the medicament reservoir 4, the displacement is completed here, and the arrangement shown in FIG. 1 is obtained, for example. In the second and the third embodiments of the medicament reservoir 4, pivoting of the medicament reservoir 4 is still required so that the first web 25 reaches the first axial clearance 7a and the second web 26 reaches the second axial clearance 7b (cf. FIG. 8). It is conceivable here that the displacement direction 15 is disposed parallel to the optical axis 11, see FIG. 8.

The medicament reservoir 4 can include only a single material which includes the medicament, as is the case in the first embodiment according to the disclosure of the medicament reservoir 4 according to FIG. 3.

Alternatively, it is conceivable that the medicament reservoir 4 includes a first material which includes the medicament, and a second material which is different from the first material and forms the first web 25, as is the case in the second embodiment according to the disclosure of the medicament reservoir 4 according to FIG. 4. For this purpose, the medicament reservoir 4 can have a first cover plate 21 which is formed of the second material and forms the first end face 31. It is also conceivable that the second material forms the second web 26. For this purpose, the medicament reservoir 4 can have a second cover plate 22, which is formed of the second material and forms the second end face 32. A central part 20, which is formed of the first material and in particular forms the circumferential surface 33, can be disposed between the first cover plate 21 and the second cover plate 22.

The first cover plate 21 and/or the second cover plate 22 can be porous. This allows the medicament to be released more quickly.

The medicament reservoir 4 can have a column extending through the first material and connecting the first cover plate 21 and the second cover plate 22 to one another. For example, the column can include the second material and/or consist of the second material. It is also conceivable that a plurality of columns are provided, which in particular all connect the first cover plate 21 and the second cover plate 22 to one another.

It is understood that the foregoing description is that of the preferred embodiments of the invention and that various changes and modifications may be made thereto without departing from the spirit and scope of the invention as defined in the appended claims.

### LIST OF REFERENCE SIGNS

1 Intraocular lens
2 Optical body
3 Haptic arm
4 Medicament reservoir
5 Capsular bag
6 Radial clearance
7a First axial clearance
7b Second axial clearance
8 Through-hole
9 Longitudinal end
10 Radial protrusion
11 Optical axis
12 Axial direction
13 Radial direction
14 Circumferential direction
15 Displacement direction
16 Medicament reservoir radial direction
17 Medicament reservoir circumferential direction
20 Central part
21 First cover plate
22 Second cover plate
23 First medicament reservoir clearance
24 Second medicament reservoir clearance
25 First web
26 Second web
31 First end face
32 Second end face
33 Circumferential surface
34 First longitudinal end
35 Second longitudinal end
41 First flank of the radial clearance
42 Second flank of the radial clearance
43 Third flank of the radial clearance
44 First flank of the first axial clearance
45 Second flank of the first axial clearance
46 Third flank of the first axial clearance
47 First flank of the second axial clearance
48 Second flank of the second axial clearance
49 Third flank of the third axial clearance

1. A medicament reservoir for an intraocular lens, the medicament reservoir having a medicament and comprising:
a through-hole;
a first medicament reservoir clearance communicating with said through-hole;
a first web delimiting said first medicament reservoir clearance;
a first end face delimiting said through-hole;
a second end face disposed to face away from said first end face and to delimit said through-hole;
said through-hole defining a first longitudinal end in a region of said first end face and defining a second longitudinal end in a region of said second end face;
said medicament reservoir defining a displacement direction oriented from said first longitudinal end to said second longitudinal end; and,
said first medicament reservoir clearance being configured so as to cause said through-hole to be accessible from outside said medicament reservoir via said first medicament reservoir clearance in a medicament reservoir radial direction in terms of said displacement direction.

2. The medicament reservoir of claim 1, wherein said first web forms part of said first end face.

3. The medicament reservoir of claim 1, wherein said second end face is not formed in a region of said first medicament reservoir clearance, so that said first medicament reservoir clearance is accessible from outside said medicament reservoir in said displacement direction.

4. The medicament reservoir of claim 1, further comprising:
a second medicament reservoir clearance communicating with said through-hole;
a second web delimiting said second medicament reservoir clearance; and,
said second medicament reservoir clearance being formed so as to cause said through-hole to be accessible from outside of said medicament reservoir via said second medicament reservoir clearance in a further medicament reservoir radial direction in terms of said displacement direction.

5. The medicament reservoir of claim 4, wherein said second web forms part of said second end face.

6. The medicament reservoir of claim 4, wherein said first end face is not formed in a region of said second medicament reservoir clearance so as to permit said second medicament reservoir clearance to be accessible from outside said medicament reservoir counter to said displacement direction.

7. The medicament reservoir of claim 1, wherein said medicament reservoir comprises only a single material which comprises the medicament.

8. The medicament reservoir of claim 1, wherein said medicament reservoir comprises a first material which comprises the medicament and a second material different from said first material and forms said first web.

9. The medicament reservoir of claim 8, wherein said medicament reservoir has a first cover plate formed by said second material and forms said first end face.

10. An intraocular lens comprising:
an optical body;
a haptic arm attached to said optical body; and,
a medicament reservoir including:
a through-hole;
a first medicament reservoir clearance communicating with said through-hole;
a first web delimiting said first medicament reservoir clearance;
a first end face delimiting said through-hole;
a second end face disposed to face away from said first end face and to delimit said through-hole;
said through-hole defining a first longitudinal end in a region of said first end face and a second longitudinal end in a region of said second end face;
said medicament reservoir defining a displacement direction oriented from said first longitudinal end to said second longitudinal end;
said first medicament reservoir clearance being configured so as to cause said through-hole to be accessible from outside said medicament reservoir via said first medicament reservoir clearance in a medicament reservoir radial direction in terms of said displacement direction; and,
said haptic arm being disposed in said through-hole and disposed in said first medicament reservoir clearance.
